(19) United States
(12) Patent Application Publication
(10) Pub. No.: US 2007/0088299 A1
(43) Pub. Date: Apr. 19, 2007

(54) Emesis kit

(76) Inventor: Emily Ayre, Sandy, UT (US)

Correspondence Address: STOEL RIVES LLP - SLC, 201 SOUTH MAIN STREET, ONE UTAH CENTER, SALT LAKE CITY, UT, 84111, US

(21) Appl. No.: 11/252,112

(22) Filed: Oct. 17, 2005

(51) Int. Cl. A61M 1/00

(52) U.S. Cl. 604/317; 206/438

(57) ABSTRACT

Disclosed are embodiments of an emesis kit and the components thereof. Some embodiments of the invention consist of a self-contained kit including an emesis collection device, such as an emesis collection bag, and various other items that may be useful to a person who has vomited, such as items for cleaning up the vomit, items for freshening the user's breath, personal hygiene items, and items to help prevent further emesis, such as anti-nausea items.

### TECHNICAL FIELD

The present invention relates to emesis kits and the individual components of emesis kits.

### BRIEF DESCRIPTION OF THE DRAWINGS

Understanding that drawings depict only certain preferred embodiments of the invention and are therefore not to be considered limiting of its scope, the preferred embodiments will be described and explained with additional specificity and detail through the use of the accompanying drawings in which:

FIG. 1 is a perspective view of an embodiment of an emesis collection bag.

FIG. 2 is a perspective view of an embodiment of an emesis collection bag including a handle.

FIG. 3 is a side elevation view of an embodiment of an emesis kit including an emesis collection bag and a clean-up packet.

FIG. 4 is a schematic view of an embodiment of a clean-up packet.

### DETAILED DESCRIPTION OF PREFERRED EMBODIMENTS

Described below are various embodiments of emesis collection devices, emesis kits, and emesis kit components. In the following description, numerous specific details are provided for a thorough understanding of the embodiments of the invention. However, those skilled in the art will recognize that the invention can be practiced without one or more of the specific details, or with other methods, components, materials, etc.

In addition, in some cases, well-known structures, materials, or operations are not shown or described in detail in order to avoid obscuring aspects of the invention. Furthermore, the described features, structures, or characteristics may be combined in any suitable manner in one or more embodiments.

Certain preferred embodiments and implementations will now be described.

One embodiment of the invention is an emesis clean-up kit. The kit is comprised of an emesis collection device, and some embodiments of the kit include attached personal clean-up items. One embodiment of the emesis collection device comprises a bag made of a plastic with three closed, sealed sides and a top side which is open for insertion of one's face when one needs to vomit. In certain embodiments of the device, the plastic may be a plastic having a thickness of about 2-3 mm. The device itself may be sealable, to ensure that vomit or other bodily fluids deposited therein are securely stored until disposal of the device and accompanying clean-up items.

In embodiments including clean-up items, the clean-up items may be sealed together in a sterile package and attached to the emesis collection device so that the clean-up packet can be easily pulled from the collection device at the time of use, and clean-up items used after vomiting. The clean-up packet may include, for example, various dry and moist towelettes, items to freshen one's breath, anti-naseau items, and/or personal hygiene items. Such items may include, but are not limited to, mints, self-pasting/disposable toothbrushes, finger tooth brushes, gum, suckers, breath strips, etc. The clean-up packet that is included with some embodiments of the invention may include one or more (or all) of the above items.

The emesis kit may be self-contained, individually packaged, foldable and/or easily transportable for use by anyone, anywhere it is needed.

In one particular embodiment of the emesis collection device that may be included with embodiments of the emesis kit, the collection device is a bag that is approximately 8.75 inches tall and 5.75 inches wide. The bag may also be gusseted in some embodiments, and in one particular embodiment the bag is a three-inch gusseted bag. Of course, in other embodiments, the emesis collection device may comprise a structure other than a bag. Any structure capable of receiving and holding vomit may be used as an emesis collection device.

In some embodiments, the bag may be made of plastic, and my also have a sticky, peelable label at the top. The label in some embodiments may be approximately ½ to ¾ of an inch down from the top of bag, allowing for easy peel and seal capability.

To place the items into a kit format, the bag or other collection device may be folded into thirds, and the clean-up packet attached with a light adhesive. In some embodiments, the packet may be placed against the bottom of the bag, so that when the emesis collection device is folded or rolled, the clean-up packet is enveloped in the device. In other embodiments, the clean-up packet or such items may be packaged together in with the bag by stapling, binding, or otherwise attaching the packet or individual items to the bag. Alternatively, the packet or individual items may be placed inside the bag. The whole kit may be packaged in, for example, a polypropelene bag or packaging. In one embodiment, the kit is approximately 3 inches tall, 5.75 inches wide, and ½ inch to 1 inch thick when packaged. Of course, any number of bags or other collection devices of other sizes, shapes, dimensions, materials, and other characteristics may be used in other embodiments. The polypropelene or other such packaging may be placed around the whole kit so that the kit's contents will remain clean and untouched until opened for use. The collection device and/or other kit components in some embodiments may be held together by an elastic band or other “tie” mechanism inside the outer packaging.

The emesis collection component/bag of the kit may be manufactured using 2-3 mm thick plastic, and may be a sealable bag. The bag may have printing on one or more sides, including the front, back, inner gussets, and bottom of the bag. The printed information may include instructions for use, legal disclaimers, name of the product, company contact information, uses of the product, etc. In some embodiments of an emesis collection bag, the bag may also have at least one handle to facilitate holding the collection bag. The handles may extend from, for example, opposing portions of the bag. In some embodiments, the handles may extend from opposing portions of the top of the bag. Such handles may also be used to facilitate opening and holding the bag in a suitable position for use.

The clean-up packs may include, but are not limited to the following items:

1. Dry towelettes. Some suitable dry towelettes may be approximately fifteen inches by fifteen inches and may be made of a disposable, paper-like substance, much like a napkin or paper towel. In one specific embodiment, dry towelettes measuring approximately sixteen inches by sixteen inches may be used. In another specific embodiment, towelettes measuring approximately ten inches by ten inches may be used. This will provide the user with a way to clean off his or her face, hands, and other soiled body parts and/or items/areas.

2. Moist towelettes. Some suitable moist towelettes may measure approximately eight inches by eight inches and may be manufactured, folded, and pre-packaged in a paper packet. Of course, other sizes of towelettes may be used.

3. Breathfreshening and/oranti-nausea item(s). Such items may include mints, gum, breath strips, disposable toothbrushes, and/or anti-nausea suckers, etc. In some embodiments, such items may comprise peppermint and/or ginger, for example, which are known to help reduce nausea in many people, and can also help rid the user of the bad taste in their mouth following the act of vomiting.

In some embodiments, the kit may also include gloves, such as rubber gloves, to further assist the user in cleaning up.

In some embodiments, the emesis collection kit will be light, easily transportable, and disposable so that it can be easily transported in any purse, carry on bag, automobile, etc. Some such kits may be small enough to fit in a pant pocket or other pocket.

In some embodiments, once the kit has been opened and used, all used clean-up items can be fit into the emesis collection device and be sealed therein prior to disposal.

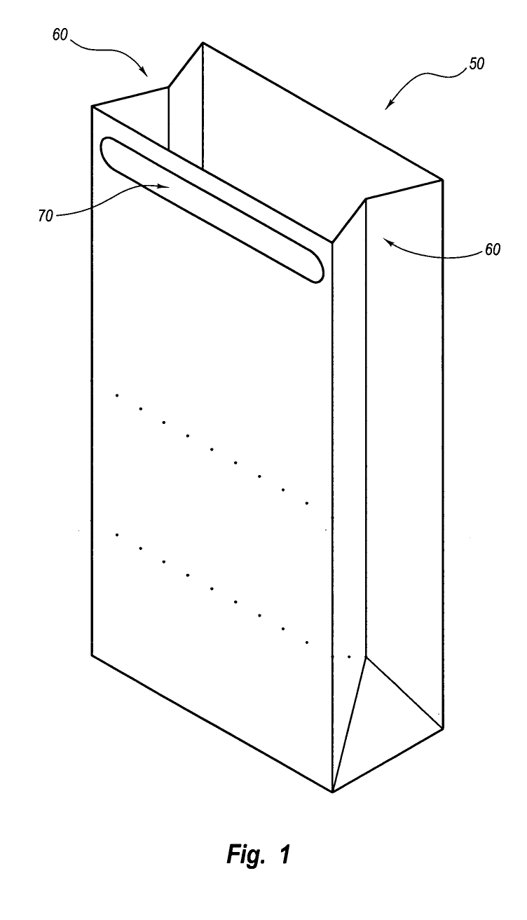

With reference now to the accompanying figures, one illustrative embodiment will now be shown and described. FIG. 1 depicts an emesis collection bag 50. Emesis collection bag 50 includes gussets 60, which are formed on opposing sides of the bag and which may allow the bag 50 to be stored in a compact configuration and be expanded for or upon use. In some embodiments, the gussets may also be configured to allow the bag to be opened and stood up on a floor or other flat surface. The gussets in such embodiments may facilitate maintaining the bag in a upright and open position without being held by a user.

Emesis collection bag 50 may also include a sticky region 70. Sticky region 70 may allow the bag to be folded and adhered to itself to seal in vomit, soiled clean-up items and associated odors before disposal of used kit. This is one example of a structure used to provide a sealable emesis collection device. In some preferred embodiments, sticky region 70 may be covered prior to use with a removable label. After using the emesis collection device, the user may then remove the label, fold the top portion of the bag over on top of itself, and use the sticky region to provide a seal to keep the bag or other device closed.

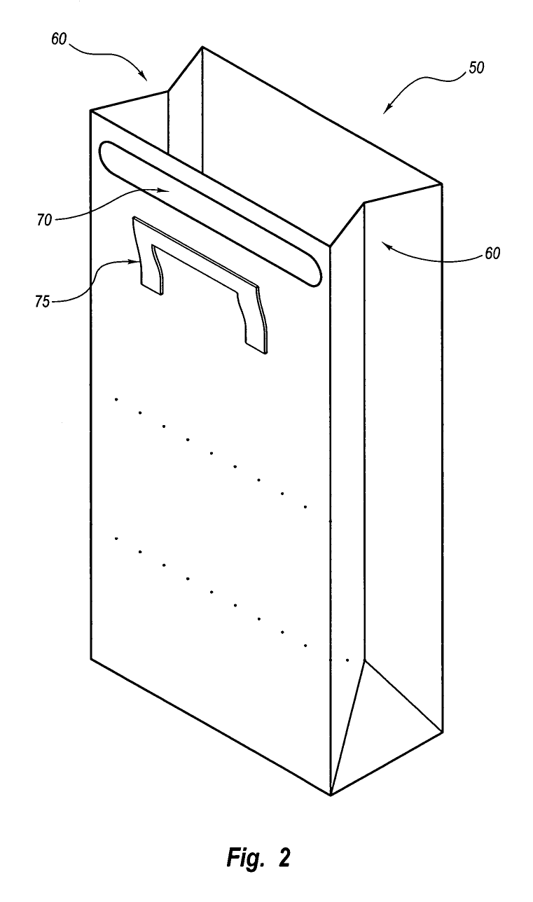

FIG. 2 depicts an alternative emesis collection bag including a handle 75. Handle 75 comprises a length of material formed into a loop and attached to the side of the bag. Of course, countless variations in the size, shape, and configuration of the handle(s) are possible. For example, two handles placed on opposing sides of the bag may be used, the opening(s) could be smaller, larger, or shaped differently, etc. As another alternative, the handles may extend or be extendable above the top of the bag, similar to a purse string. As still another alternative, the handle could comprise an opening near the top of the bag that extends along at least a portion the width of the bag on that side.

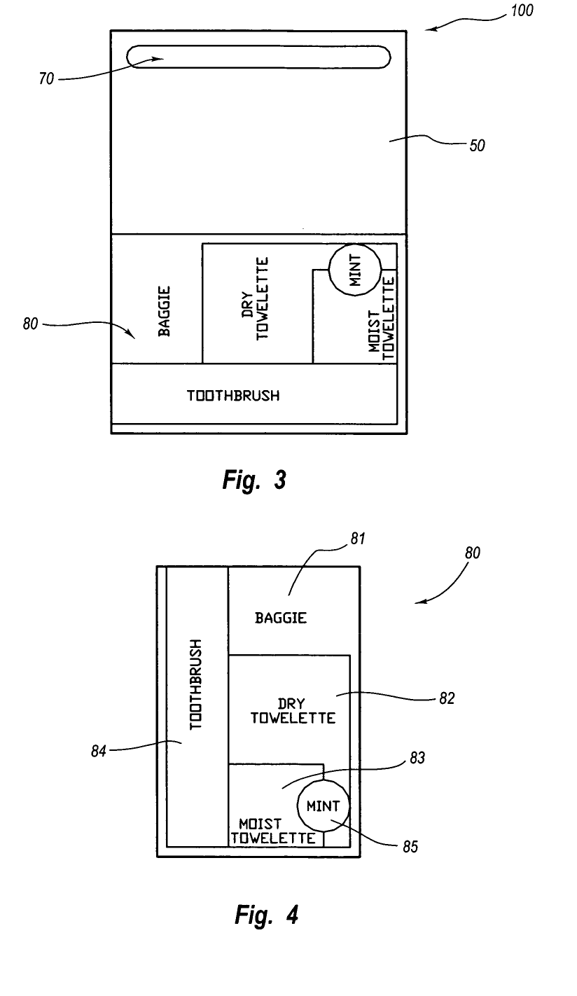

FIG. 3 depicts the emesis collection bag 50 with a clean-up packet 80 attached thereto, together comprising an emesis kit 100. Clean-up packet 80 may be attached to bag 50 by any process/structure available to one of ordinary skill in the art, such as, for example, an adhesive, staples, bands, tape, etc. The items in the clean-up packet 80 may be packaged together in a baggie 81 or other packaging material if desired. Likewise, the clean-up packet 80 may be packaged together with the collection bag 50 in a separate set of packaging material if desired. In such embodiments, the clean-up packet need not necessarily be attached to the collection bag. Instead, the clean-up packet may be connected with the collection bag by virtue of the clean-up packet being packaged with the collection bag.

In some embodiments, such as embodiments in which the clean-up packet is attached to the collection bag with a light adhesive, or in embodiments in which the clean-up packet is simply packaged with, but not attached to, the collection bag, the clean-up packet 80 may be easily removed from the collection bag, as shown in FIG. 4

As shown in FIGS. 3 and 4, and as previously discussed, the clean-up packet 80 may include items such as a dry towelefte 82, a moist towelette 83, a toothbrush 84, and a mint 85. Such items may be packaged and arranged together in any conventional manner.

The above description fully discloses the invention including preferred embodiments thereof. Without further elaboration, it is believed that one skilled in the art can use the preceding description to utilize the invention to its fullest extent. Therefore, the examples and embodiments disclosed herein are to be construed as merely illustrative and not a limitation of the scope of the present invention in any way.

It will be obvious to those having skill in the art that many changes may be made to the details of the above-described embodiments without departing from the underlying principles of the invention. The scope of the present invention should, therefore, be determined only by the following claims.

1. An emesis kit, comprising:
an emesis collecton device; and
a clean-up pack connected with the emesis collection device, wherein the clean-up pack comprises at least one item selected from the group consisting of towelettes, breath fresheners, personal hygiene items, and anti-nausea items, and wherein the emesis collection device and the clean-up pack are packaged together in a packaging material.

2. The emesis kit of claim 1, wherein the emesis collection device is sealable.

3. The emesis kit of claim 2, wherein the emesis collection device comprises a sticky region covered with a removable label, wherein, upon removal of the label, the sticky region is configured to allow the collection device to be folded and adhered to itself to seal the collection device closed.

4. The emesis kit of claim 1, wherein the emesis collection device comprises a bag.

5. The emesis kit of claim 4, wherein the emesis collection bag is gusseted.

6. The emesis kit of claim 1, wherein the clean-up pack is attached to the emesis collection device.

7. The emesis kit of claim 6, wherein the clean-up pack is attached to the emesis collection device with an adhesive.

8. The emesis kit of claim 1, wherein the clean-up pack comprises moist towelettes.

9. The emesis kit of claim 1, wherein the clean-up pack comprises at least one breath freshener, and wherein the at least one breath freshener comprises at least one item selected from the group consisting of mints and gum.

10. The emesis kit of claim 1, wherein the clean-up pack comprises at least one personal hygiene item, and wherein the at least one personal hygiene item comprises a toothbrush.

11. The emesis kit of claim 1, wherein the clean-up pack comprises at least one anti-nausea item, and wherein the at least one anti-nausea item comprises an anti-nausea sucker.

12. The emesis kit of claim 11, wherein the at least one anti-nausea item comprises at least one item selected from the group consisting of ginger and peppermint.

13. The emesis kit of claim 1, wherein the clean-up pack comprises at least two items selected from the group consisting of towelettes, breath fresheners, personal hygiene items, and anti-nausea items.

14. The emesis kit of claim 13, wherein the clean-up pack comprises towelettes and breath fresheners.

15. (canceled)

16. The emesis kit of claim 1, wherein the emesis collection device comprises at least one handle to facilitate holding the collection device.

17. The emesis kit of claim 16, wherein the emesis collection device comprises two handles extending from opposing portions of the collection device.

18. An emesis kit, comprising:
a sealable emesis collection bag; and
a clean-up pack connected with the emesis collection bag, wherein the clean-up pack comprises at least one breath freshener and at least one towelette, wherein the emesis collection device and the clean-up pack are packaged together in a packaging material.

19. The emesis kit of claim 18, wherein the clean-up pack further comprises at least one item selected from the group consisting of anti-nausea items and personal hygiene items.

20. (canceled)

21. The emesis kit of claim 18, wherein the clean-up pack is attached to the emesis collection bag.

22. An emesis kit, comprising:
an emesis collection bag, wherein the emesis collection bag is gusseted and sealable; and
a clean-up pack attached to the emesis collection bag, wherein the clean-up pack comprises moist towelettes, breath fresheners, and at least one personal hygiene item, wherein the breath fresheners comprise at least one item selected from the group consisting of mints and gum, and wherein the emesis collection device and the clean-up pack are packaged together in a packaging material.

23. The emesis kit of claim 22, wherein the at least one personal hygiene item comprises a toothbrush.

24. The emesis kit of claim 22, wherein the clean-up pack further comprises at least one anti-ausea item.
